(19) United States
(12) Patent Application Publication
(10) Pub. No.: US 2013/0157875 A1
(43) Pub. Date: Jun. 20, 2013

(54) METHODS FOR ASSESSING GENOMIC INSTABILITIES

(71) Applicant: Anthony P. Shuber, Mendon, MA (US)

(72) Inventor: Anthony P. Shuber, Mendon, MA (US)

(21) Appl. No.: 13/741,183

(22) Filed: Jan. 14, 2013

Related U.S. Application Data
(63) Continuation-in-part of application No. 13/364,840, filed on Feb. 2, 2012.
(60) Provisional application No. 61/509,898, filed on Jul. 20, 2011.

(51) Int. Cl. C12Q 1/68

(52) U.S. Cl. CPC C12Q 1/6883
USPC 506/2; 435/6.11; 506/9

(57) ABSTRACT

The invention generally relates to methods for assessing a fetal abnormality.

### RELATED APPLICATION

The present patent application is a continuation-in-part of U.S. patent application Ser. No. 13/364,840, filed Feb. 2, 2012, which claims the benefit of and priority to U.S. provisional patent application Ser. No. 61/509,898 filed on Jun. 20, 2011, the entirety of each of which is incorporated by reference herein in its entirety.

### FIELD OF THE INVENTION

The invention generally relates to methods for assessing a fetal abnormality.

### BACKGROUND

Fetal aneuploidy (e.g., Down syndrome, Edward syndrome, and Patau syndrome) and other chromosomal aberrations affect 9 of 1,000 live births (Cunningham et al. in Williams Obstetrics, McGraw-Hill, N.Y., p. 942, 2002). Chromosomal abnormalities are generally diagnosed by karyotyping of fetal cells obtained by invasive procedures such as chorionic villus sampling or amniocentesis. Those procedures are associated with potentially significant risks to both the fetus and the mother. Noninvasive screening using maternal serum markers or ultrasound are available but have limited reliability (Fan et al., PNAS, 105(42):16266-16271, 2008).

Since the discovery of intact fetal cells in maternal blood, there has been intense interest in trying to use those cells as a diagnostic window into fetal genetics (Fan et al., PNAS, 105(42):16266-16271, 2008). The discovery that certain amounts (between about 3% and about 6%) of cell-free fetal nucleic acids exist in maternal circulation has led to the development of noninvasive PCR based prenatal genetic tests for a variety of traits.

Generally, those methods look for genomic instabilities that are known to be associated with a fetal abnormality and assess a fetus based on the presence or absence of the known genomic instability. Such methods have limited diagnostic value for a large percentage of the population due to the fact that a diagnosis is based upon using only known genomic instabilities and does not account for genomic instabilities that have not previously been associated with a particular type of fetal abnormality. Additionally, such methodologies treat all genomic instabilities identically, making a diagnosis based upon presence or absence of the genomic instability while failing to account for differences in those genomic instabilities.

### SUMMARY

The invention provides methods for assessing genomic instabilities in samples including fetal nucleic in order to assess a fetus. Methods of the invention recognize that all genomic instabilities within a sample are not the same. Genomic instabilities vary in type, location, and size ranging from a discrete mutation in a single base in the DNA of a single gene to a gross chromosome abnormality involving the addition or subtraction of an entire chromosome or set of chromosomes. Moreover, genomic instabilities vary in significance and severity. Methods of the invention account for the fact that genomic instabilities vary in significance and severity and assess each genomic instability based on its own unique characteristics, which adds a level of information for in-depth prognosis not provided by previous diagnostic and prognostic methods. In this manner, methods of the invention provide a personalized assessment of risk to a fetus and options for parents.

The overall prognosis may be referred to as a coefficient of risk for prenatal diagnosis, and the output may be represented as a single value, such as a continuous from 0 (severely abnormal) to 1 (normal fetus), or 0 (severely abnormal) to 10 (normal fetus), etc.. Such value may be calculated in various different ways. In one embodiment, the coefficient of risk of the prenatal diagnosis is determined by taking the percent of altered nucleic acid and dividing it by total nucleic acid. In other embodiments, the coefficient of risk of the prenatal diagnosis is determined by taking the percent of nucleic acid changes from a reference and dividing it by a number of expected changes.

Methods of the invention account for significance and severity of each genomic instability in a nucleic acid as it relates to causing a fetal abnormality by assigning a weighted value to each genomically unstable locus in a nucleic acid sequence obtained from a sample. Assigning a weighted value allows methods of the invention to individually assess each genomic instability in the sample and to ultimately causally relate all genomic instabilities within the nucleic acid sequence to an overall prognosis for the fetus.

The weighted value may be scaled in any manner including and not limited to assigning a positive or negative integer to reflect the significance or severity of the locus as compared to a certain sequence known to be assigned with a fetal abnormality. The weighed value provides significant insight into the prognosis of the fetus because each genomically unstable locus may be factored into determining the prognosis of the fetus, instead of only identified matches to known instabilities related to fetal abnormalities. In one embodiment, a comparison of the weighted values over time and over courses of treatment allows one to alter treatment based on the specific variations of all instabilities linked to fetal abnormalities.

Methods of the invention provide for assigning weighted values to genomic instabilities within the nucleic acid even if they do not specifically match a genomic instability linked to a fetal abnormality. Weighted values may also be scaled from a normal reference sequence or from a reference sequence of a fetal abnormality. In certain embodiments, the weighted values are scaled, factoring in a suspected sequence of a fetal abnormality, in which the fetus is suspected of having an abnormality. Weighted values are assigned to genomically unstable locus based on the type, location, and amount of genetic material affected by the instability.

The genomically unstable loci may be analyzed and characterized using any criteria that allow for the fetus to be assessed. For example, each genomically unstable locus may be weighted based on the type of genomic instability found at the locus. Exemplary types of genomic instabilities include subtle sequence changes, alterations in chromosome number, translocations of chromosomes, and gene amplification. Subtle sequence changes and alterations in chromosome number may be further broken down into subtypes including but not limited to additions, deletions, and substitution. Weighted values may be based on solely on the type of change, or the weighted values may be based on comparing the type of genomic instability to a reference sequence of a fetal abnormality.

Another methodology for assigning the weighted value to each genomically unstable locus is based upon a location of the genomically unstable locus within the chromosome. For example, the weighted value may be based upon the proximity of the instability to telomeres. Alternatively, the weighted value may be based upon the proximity of the instability to known locations of genomic instability found in certain fetal abnormalities. Based on the location, the weighted value is accordingly assigned.

A weighted value may also be assigned to genomically unstable loci within the sample based on the number of nucleotides within each genomically unstable locus. In other words, the amount of genetic material in the nucleic acid affected by the instability determines the assigned weighted value. The amount of nucleotides affected may be subdivided into regions such as but not limited to coding v. non-coding and introns v. exons, and each region assigned a weighted value.

The invention further provides for categorizing the genomically unstable loci and assigning a weighted value for each category. The categories are based on the type, location, and amount of material affected, as described above. After the loci are categorized and assigned a weighted value, a weighted sum may be calculated to represent all of the genomically unstable loci within the sample. In certain embodiments, the weighted sum is the sum of each category's weighted value times the corresponding number of genomically unstable loci in each category. The invention further provides for calculating a weighted average where the weighted sum is divided by the amount of genomically unstable locus in the sample.

Any genomically unstable loci are indicative of a fetal abnormality. Generally, the greater the number of genomically unstable loci the greatly the severity of the abnormality.

Methods of the invention further provide for assessing a fetal abnormality by obtaining a sample including fetal nucleic acid, and determining a number of genomically unstable loci in the sample. With the same sample, the number of genomically stable loci is also determined and the two numbers are compared to calculate a rational number. A rational number is any number that can be expressed as the quotient or fraction a/b of two integers. The number of genomically stable loci is divided by the number of genomically unstable loci to determine the rational number. The rational number is used to assess the prognosis of the fetus. The number of genomically stable and unstable loci is determined by whole genome sequencing, targeted gene resequencing, PCR, DNA microarray, fluorescent in situ hybridization, Southern blot analysis, or Northern blot analysis.

Methods of the invention further provide for assessing the efficacy of a therapeutic treatment for the fetus by obtaining a first number of genomically unstable loci from a first sample then administering a therapeutic treatment to the patient. After the therapeutic treatment is administered, a second number of genomically unstable loci from a second sample are assessed. The difference in the first number of genomically unstable loci compared to the second number of genomically unstable loci is indicative of determining the efficacy of the therapeutic treatment. If the difference between the first and second number of genomically unstable loci is decreased, the treatment is effective while if there is an increase or no change then that therapeutic treatment is ineffective and an alternate therapeutic treatment should be considered.

Alternatively, the efficacy of the therapeutic treatment can be determined by obtaining a first rational number of the first number of genomically unstable loci compared to a first number of genomically stable loci and a second rational number of the second number of genomically unstable loci compared to a second number of genomically stable loci. The therapeutic treatment is effective if the second rational number is lesser than the first rational number, while if it is greater or there is no change an alternate therapeutic treatment should be considered.

### DETAILED DESCRIPTION

Methods of the invention use genomically unstable loci to assess fetal abnormalities. Samples having fetal nucleic acid are obtained. In certain embodiments, methods of the invention account for significance and severity of each genomic instability in a nucleic acid as it relates to causing a fetal abnormality by assigning a weighted value to each genomically unstable locus in a nucleic acid sequence obtained from a sample. Assigning a weighted value allows methods of the invention to individually assess each genomic instability in the sample and to ultimately causally relate all genomic instabilities within the nucleic acid sequence to an overall assessment of the fetus, i.e., the coefficient of risk for prenatal diagnosis.

The coefficient of risk for prenatal diagnosis the output may be represented as a single value, such as a continuous from 0 (severely abnormal) to 1 (normal fetus), or 0 (severely abnormal) to 10 (normal fetus), etc.. Such value may be calculated in various different ways. In one embodiment, the coefficient of risk of the prenatal diagnosis is determined by taking the percent of altered nucleic acid and dividing it by total nucleic acid. In other embodiments, the coefficient of risk of the prenatal diagnosis is determined by taking the percent of nucleic acid changes from a reference and dividing it by a number of expected changes.

A variety of genetic abnormalities may be detected according to the present methods, including aneuplody (i.e., occurrence of one or more extra or missing chromosomes) or known alterations in one or more genes, such as, CFTR, Factor VIII (F8 gene), beta globin, hemachromatosis, G6PD, neurofibromatosis, GAPDH, beta amyloid, and pyruvate kinase. The sequences and common mutations of those genes are known. Other genetic abnormalities may be detected, such as those involving a sequence which is deleted in a human chromosome, is moved in a translocation or inversion, or is duplicated in a chromosome duplication, in which the sequence is characterized in a known genetic disorder in the fetal genetic material not present in the maternal genetic material. For example chromosome trisomies may include partial, mosaic, ring, 18, 14, 13, 8, 6, 4 etc. A listing of known abnormalities may be found in the OMIM Morbid map, http://www.ncbi.nlm.nih.gov/Omim/getmorbid.cgi, the contents of which are incorporated by reference herein in their entirety.

These genetic abnormalities include mutations that may be heterozygous and homozygous between maternal and fetal nucleic acid, and to aneuploidies. For example, a missing copy of chromosome X (monosomy X) results in Turner's Syndrome, while an additional copy of chromosome 21 results in Down Syndrome. Other diseases such as Edward's Syndrome and Patau Syndrome are caused by an additional copy of chromosome 18, and chromosome 13, respectively. The present method may be used for detection of a translocation, addition, amplification, transversion, inversion, aneuploidy, polyploidy, monosomy, trisomy, trisomy 21, trisomy 13, trisomy 14, trisomy 15, trisomy 16, trisomy 18, trisomy 22, triploidy, tetraploidy, and sex chromosome abnormalities including but not limited to XO, XXY, XYY, and XXX.

Examples of diseases where the target sequence may exist in one copy in the maternal DNA (heterozygous) but cause disease in a fetus (homozygous), include sickle cell anemia, cystic fibrosis, hemophilia, and Tay Sachs disease. Accordingly, using the methods described here, one may distinguish genomes with one mutation from genomes with two mutations.

Sickle-cell anemia is an autosomal recessive disease. Nine-percent of US African Americans are heterozygous, while 0.2% are homozygous recessive. The recessive allele causes a single amino acid substitution in the beta chains of hemoglobin.

Tay-Sachs Disease is an autosomal recessive resulting in degeneration of the nervous system. Symptoms manifest after birth. Children homozygous recessive for this allele rarely survive past five years of age. Sufferers lack the ability to make the enzyme N-acetyl-hexosaminidase, which breaks down the GM2 ganglioside lipid.

Another example is phenylketonuria (PKU), a recessively inherited disorder whose sufferers lack the ability to synthesize an enzyme to convert the amino acid phenylalanine into tyrosine Individuals homozygous recessive for this allele have a buildup of phenylalanine and abnormal breakdown products in the urine and blood.

Hemophilia is a group of diseases in which blood does not clot normally. Factors in blood are involved in clotting. Hemophiliacs lacking the normal Factor VIII are said to have Hemophilia A, and those who lack Factor IX have hemophilia B. These genes are carried on the X chromosome, so sequencing methods of the invention may be used to detect whether or not a fetus inherited the mother's defective X chromosome, or the father's normal allele.

### Samples

Methods of the invention involve obtaining a sample, e.g., a tissue or body fluid, that is suspected to include fetal nucleic acids. Such samples may include saliva, urine, tear, vaginal secretion, amniotic fluid, breast fluid, breast milk, sweat, or tissue. In certain embodiments, this sample is drawn maternal blood, and circulating DNA is found in the blood plasma, rather than in cells. A preferred sample is maternal peripheral venous blood.

In certain embodiments, approximately 10-20 mL of blood is drawn. That amount of blood allows one to obtain at least about 10,000 genome equivalents of total nucleic acid (sample size based on an estimate of fetal nucleic acid being present at roughly 25 genome equivalents/mL of maternal plasma in early pregnancy, and a fetal nucleic acid concentration of about 3.4% of total plasma nucleic acid). However, less blood may be drawn for a genetic screen where less statistical significance is required, or the nucleic acid sample is enriched for fetal nucleic acid.

Because the amount of fetal nucleic acid in a maternal sample generally increases as a pregnancy progresses, less sample may be required as the pregnancy progresses in order to obtain the same or similar amount of fetal nucleic acid from a sample.

### Enrichment

In certain embodiments, the sample (e.g., blood, plasma, or serum) may optionally be enriched for fetal nucleic acid by known methods, such as size fractionation to select for DNA fragments less than about 300 bp. Alternatively, maternal DNA, which tends to be larger than about 500 bp, may be excluded.

In certain embodiments, the maternal blood may be processed to enrich the fetal DNA concentration in the total DNA, as described in Li et al., J. Amer. Med. Assoc. 293:843-849, 2005), the contents of which are incorporated by reference herein in their entirety. Briefly, circulatory DNA is extracted from 5 mL to 10 mL maternal plasma using commercial column technology (Roche High Pure Template DNA Purification Kit; Roche, Basel, Switzerland) in combination with a vacuum pump. After extraction, the DNA is separated by agarose gel (1%) electrophoresis (Invitrogen, Basel, Switzerland), and the gel fraction containing circulatory DNA with a size of approximately 300 by is carefully excised. The DNA is extracted from this gel slice by using an extraction kit (QIAEX II Gel Extraction Kit; Qiagen, Basel, Switzerland) and eluted into a final volume of 40 μL sterile 10-mM trishydrochloric acid, pH 8.0 (Roche).

DNA may be concentrated by known methods, including centrifugation and various enzyme inhibitors. The DNA is bound to a selective membrane (e.g., silica) to separate it from contaminants. The DNA is preferably enriched for fragments circulating in the plasma, which are less than 1000 base pairs in length, generally less than 300 bp. This size selection is done on a DNA size separation medium, such as an electrophoretic gel or chromatography material. Such a material is described in Huber et al. (Nucleic Acids Res. 21(5):1061-1066, 1993), gel filtration chromatography, TSK gel, as described in Kato et al., (J. Biochem, 95(1):83-86, 1984). The content of each of these references is incorporated by reference herein in their entirety.

In addition, enrichment may be accomplished by suppression of certain alleles through the use of peptide nucleic acids (PNAs), which bind to their complementary target sequences, but do not amplify.

Plasma RNA extraction is described in Enders et al. (Clinical Chemistry 49:727-731, 2003), the contents of which are incorporated by reference herein in their entirety. As described there, plasma harvested after centrifugation steps is mixed with Trizol LS reagent (Invitrogen) and chloroform. The mixture is centrifuged, and the aqueous layer transferred to new tubes. Ethanol is added to the aqueous layer. The mixture is then applied to an RNeasy mini column (Qiagen) and processed according to the manufacturer's recommendations.

Another enrichment step may be to treat the blood sample with formaldehyde, as described in Dhallan et al. (J. Am. Med. Soc. 291(9): 1114-1119, March 2004; and U.S. patent application number 20040137470), the contents of each of which are incorporated by reference herein in their entirety. Dhallan et al. (U.S. patent application number 20040137470) describes an enrichment procedure for fetal DNA, in which blood is collected into 9 ml EDTA Vacuette tubes (catalog number NC9897284) and 0.225 ml of 10% neutral buffered solution containing formaldehyde (4% w/v), is added to each tube, and each tube gently is inverted. The tubes are stored at 4° C. until ready for processing.

Agents that impede cell lysis or stabilize cell membranes can be added to the tubes including but not limited to formaldehyde, and derivatives of formaldehyde, formalin, glutaraldehyde, and derivatives of glutaraldehyde, crosslinkers, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, cleavable crosslinkers, etc. Any concentration of agent that stabilizes cell membranes or impedes cell lysis can be added. In certain embodiments, the agent that stabilizes cell membranes or impedes cell lysis is added at a concentration that does not impede or hinder subsequent reactions.

Flow cytometry techniques can also be used to enrich fetal cells (Herzenberg et al., PNAS 76:1453-1455, 1979; Bianchi et al., PNAS 87:3279-3283, 1990; Bruch et al., Prenatal Diagnosis 11:787-798, 1991). Saunders et al. (U.S. Pat. No. 5,432,054) also describes a technique for separation of fetal nucleated red blood cells, using a tube having a wide top and a narrow, capillary bottom made of polyethylene. Centrifugation using a variable speed program results in a stacking of red blood cells in the capillary based on the density of the molecules. The density fraction containing low-density red blood cells, including fetal red blood cells, is recovered and then differentially hemolyzed to preferentially destroy maternal red blood cells. A density gradient in a hypertonic medium is used to separate red blood cells, now enriched in the fetal red blood cells from lymphocytes and ruptured maternal cells. The use of a hypertonic solution shrinks the red blood cells, which increases their density, and facilitates purification from the more dense lymphocytes. After the fetal cells have been isolated, fetal DNA can be purified using standard techniques in the art.

Further, an agent that stabilizes cell membranes may be added to the maternal blood to reduce maternal cell lysis including but not limited to aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188.

An example of a protocol for using this agent is as follows: The blood is stored at 4° C. until processing. The tubes are spun at 1000 rpm for ten minutes in a centrifuge with braking power set at zero. The tubes are spun a second time at 1000 rpm for ten minutes. The supernatant (the plasma) of each sample is transferred to a new tube and spun at 3000 rpm for ten minutes with the brake set at zero. The supernatant is transferred to a new tube and stored at −80° C. Approximately two milliliters of the “buffy coat,” which contains maternal cells, is placed into a separate tube and stored at −80° C.

Genomic DNA may be isolated from the plasma using the Qiagen Midi Kit for purification of DNA from blood cells, following the manufacturer's instructions (QIAmp DNA Blood Midi Kit, Catalog number 51183). DNA is eluted in 100 μl of distilled water. The Qiagen Midi Kit also is used to isolate DNA from the maternal cells contained in the “buffy coat.”

### Extraction

Nucleic acids may be obtained by methods known in the art. Generally, nucleic acids can be extracted from a biological sample by a variety of techniques such as those described by Maniatis, et al., Molecular Cloning: A Laboratory Manual, Cold Spring Harbor, N.Y., pp. 280-281, (1982), the contents of which is incorporated by reference herein in its entirety.

It may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl₃ to denature any contaminating and potentially interfering proteins. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

### Sequencing

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes.

A sequencing technique that can be used in the methods of the provided invention includes, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using a electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

Additional detection methods can utilize binding to microarrays for subsequent fluorescent or non-fluorescent detection, barcode mass detection using a mass spectrometric methods, detection of emitted radiowaves, detection of scattered light from aligned barcodes, fluorescence detection using quantitative PCR or digital PCR methods.

### Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

A listing of gene mutations for which the present methods may be adapted is found at http://www.gdb.org/gdb, The GDB Human Genome Database, The Official World-Wide Database for the Annotation of the Human Genome Hosted by RTI International, N.C. USA.

A listing of known abnormalities may be found in the OMIM Morbid map, http://www.ncbi.nlm.nih.gov/Omim/getmorbid.cgi, the contents of which are incorporated by reference herein in their entirety.

### Other Methods of the Invention

Other techniques allowing for the detection of a nucleic acid in a sample can be used in the present invention, such as, for example, Northern blot, selective hybridization, the use of supports coated with oligonucleotide probes, amplification of the nucleic acid by RT-PCR, quantitative PCR or ligation-PCR, etc. These methods can include the use of a nucleic acid probe (for example an oligonucleotide) that can selectively or specifically detect the target nucleic acid in the sample. Chromosome specific primers are shown in Hahn et al. (U.S. patent application number 2005/0164241) hereby incorporated by reference in its entirety. Primers for the genes may be prepared on the basis of nucleotide sequences obtained from databases such as GenBank, EMBL and the like. For example, there are more than 1,000 chromosome 21 specific primers listed at the NIH UniSTS web site, which can be located at http://www.ncbi.nlm.nih.gov/entrez/query.fcgi?db=unists.

Amplification is accomplished according to various methods known to the person skilled in the art, such as PCR, LCR, transcription-mediated amplification (TMA), strand-displacement amplification (SDA), NASBA, the use of allele-specific oligonucleotides (ASO), allele-specific amplification, Southern blot, single-strand conformational analysis (SSCA), in-situ hybridization (e.g., FISH), migration on a gel, heteroduplex analysis, etc. If necessary, the quantity of nucleic acid detected can be compared to a reference value, for example a median or mean value observed in patients who do not have the abnormality, or to a value measured in parallel in a normal sample. Thus, it is possible to demonstrate a variation in the level of expression.

### Determining Presence and Type of Genomically Unstable Loci

As described above, look up tables can be used to compare sequencing results to determine genomically unstable loci of the sequence. Once a genomic sequence from one sample has been determined by sequencing, as described above, hybridization techniques are used to determine variations in sequence between the sample sequence and a reference sequence. The variations between the two sequences are the genomically unstable loci of interest.

The number of genomically unstable loci are quantified for the sample and compared to that of a reference sequence in order to assess the fetus. An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from normal fetus tissue, or from normal tissue taken from one of the parents, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular fetal abnormalities can also assess the fetus. One method of determining the presence of predetermined genomically unstable loci includes PCR. Methods for implementing PCR are well-known. In the present invention, fetal DNA fragments are amplified using human-specific primers. Amplicon of greater than about 200 by produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

### Quantitative Assessment of Genomically Unstable Loci

In certain aspects of the invention, a rational number is determined to assess the fetus. A rational number is any number that can be expressed as the quotient or fraction a/b of two integers. In this aspect, the number of genomically unstable loci is determined in a sample using methods described above. Further, the number of genomically stable loci from the same sample is determined and a ratio of the number of genomically unstable loci to genomically stable loci provides a rational number for that patient. As discussed above, any genomic instability is indicative of fetal abnormalities. There is a linear relationship between the rational number and the severity of abnormality in the fetus, the higher the number, the worse the prognosis for the fetus.

Based upon the determination of the number of genomically unstable loci and/or the presence of predetermined genomically unstable loci, methods of the invention also include providing targeted therapeutic treatment based upon the presence and/or quantity of genomically unstable loci in a sample.

### Qualitative Assessment of Genomically Unstable Loci

Certain embodiments of the invention provide for assessing fetal abnormalities through a qualitative assessment of genomically unstable loci. The qualitative assessment includes identifying the genomically unstable loci and assigning a weighted value to each genomically unstable locus. Methods of the invention provide for identifying genomically unstable loci by the type of genomic instability, the location of the genomic instability, the amount of genomic material perturbed by the genomic instability, or a combination thereof. Methods of the inventions may be used to profile genomically unstable loci causally related to fetal abnormalities generally by comparing the sample to multiple references. Alternatively, methods of the invention also provide for profiling genomically unstable loci causally related to a specific fetal abnormality a fetus is suspected of having by comparing the genomically unstable loci from a sample to a fetal abnormality reference. Embodiments further provides for assigning weighted values dependant on the identification step.

The genomically unstable loci can be identified by methods described above. Briefly, look up tables can be used to compare sequencing results to determine genomically unstable loci of the sequence. Once a genomic sequence from one sample has been determined by sequencing, hybridization techniques are used to determine variations in sequence between the sample sequence and a reference sequence. The variations between the two sequences are the genomically unstable loci of interest and the type, location, and amount of genetic material affected can also be identified from the variations.

An example of a suitable hybridization technique involves the use of DNA chips (oligonucleotide arrays), for example, those available from Affymetrix, Inc. Santa Clara, Calif. Reference sequences for use in comparison to the sample sequence include, but are not limited to, a sample from a normal tissue taken from the a fetus or a aample from a normal tissue taken from a parent, such as a buccal swab, or a human consensus sequence.

In other embodiments of the invention, a primer with predetermined genomically unstable loci that binds to the nucleic acid of the sample is indicative of that genomically unstable locus. The presence of specific genomically unstable loci in particular fetal abnormalities can also determine the prognosis of the fetus. One method of determining the presence of predetermined genomically unstable loci includes PCR. Methods for implementing PCR are well-known. In the present invention, fetal DNA fragments are amplified using human-specific primers. Amplicon of greater than about 200 by produced by PCR represents a positive screen. Other amplification reactions and modifications of PCR, such as ligase chain reaction, reverse-phase PCR, Q-PCR, and others may be used to produce detectable levels of amplicon. Amplicon may be detected by coupling to a reporter (e.g. fluorescence, radioisotopes, and the like), by sequencing, by gel electrophoresis, by mass spectrometry, or by any other means known in the art, as long as the length, weight, or other characteristic of the amplicons identifies them by size.

After determining the presence and identity of genomically unstable loci, methods of the invention provide for assigning a weighted value to each genomic instability. The weighted value is based upon a characteristic of the genomic instability, such as the type, location, amount of genetic material affect, or a combination thereof.

Methods of the invention further provide for qualitatively assessing the entire sample with a weighted sum. In such an embodiment, the genomic instabilities are characterized by type, location, or amount of nucleotides affected and each category is assigned a weighted value. A weighted sum is then derived by multiplying each category's weighted value times the number of genomically unstable loci within the category. A weighted average may further be calculated by dividing the weighted sum by a total amount of genomically unstable loci in each category.

In embodiments of the invention, the weighted value may be any integer or identifier based on the significance and severity of the genomically unstable locus. The weighted value acts as a way to scale and score genomically unstable loci in comparison to a normal reference sequence and fetal abnormality references. Certain embodiments provide for comparing the sequence to a fetal abnormality reference sequence in order to scale the sample sequence in comparison to known instabilities found in the fetal abnormality reference. The invention embodies any method of scoring or scaling.

In certain embodiments, the weighted value for instabilities may be on a scale from −10 to +10. The +10 may indicate the genomically unstable locus is extremely unstable because its an exact match to instabilities found in highly progressed or developed fetal abnormalities. A +4 may represent a genomically unstable locus that is a latent instability, meaning it will not cause lead to a fetal abnormality on its own, but may become problematic upon influence of external factors such as aging and smoking. Whereas +2 may represent a genomically unstable locus found in some undeveloped fetal abnormalities but nothing directly relates the locus to fetal abnormality progression. A 0 on the scale may include instabilities not yet known to have any effect or any negative effect towards fetal abnormalities. A −10 may include genomically unstable locus shown not to affect fetal abnormalities, for example the instability relates to learning disabilities. Further, embodiments provide for the weighted scale to include a +1 for loci that are the same as those found in fetal abnormalities, 0.5 for loci similar to those found in fetal abnormalities, and 0 for loci without a causal link to fetal abnormalities.

In certain embodiments, methods of the invention assign a weighted value based upon the type of genomically unstable locus. The main types of genomic instabilities include subtle sequence changes, alterations in chromosome number, translocations of chromosomes, and single nucleotide polymorphisms. It is recognized that genomic instabilities are linked to fetal abnormalities.

Genomic subtle sequence changes include additions, deletions, inversions, and substitutions of one or more nucleotides within a sequence, but not to the extent of large chromosomal sequence changes. A single nucleotide polymorphism (SNP) is a type of genomic subtle sequence change that occurs when a single nucleotide replaces another within the sequence. Alterations in chromosome numbers include additions, deletions, inversions and substitutions of chromosomes within a sequence. Chromosome translocation occurs when a segment of a chromosome attaches, or fuses, to another chromosome, or when noncontiguous segments within a single chromosome fuse. The result of chromosome translocation is the fusion of two different genes, in which the fused genes may have fetal abnormality causing properties or the translocation results in the disruption or deregulation of normal gene function. Gene amplifications results when multiple copies of a chromosomal segment are reproduced, instead of a single copy.

After identifying the type of genomically unstable locus, methods of the invention provide for assigning a weighted value to each genomically unstable locus. In certain embodiments, if an addition, deletion, substitution, translocation, inversion, amplification, or single nucleotide polymorphism found in the sample is similar or identical to the same type of instability in a fetal abnormality, then a weighted value reflecting its significance and severity will be assigned according. For example, consider a nucleic acid sequence with a genomically unstable locus representing an addition X, genomically unstable locus representing a translocation Y, and a genomically unstable locus representing a SNP. Both the addition, the SNP and the translocation are assigned a weighted value. If the addition X in the sample is exactly the same as an addition X found in fetal abnormality X, then addition X will receive a high value, such as +10. If the translocation Y is not yet identified as an exact translocation found in fetal abnormality sequences, but is very similar to a translocation Z found in a particular fetal abnormality, then the value of the instability will be high, such as a +6, but not as high as addition X, which represented an exact match. If the SNP Y is not found in fetal abnormalities, then its weighted value may be a 0, or if the SNP Y is identified as a harmless SNP then its weighted value will be −8. The assigned values are aggregated to arrive at a score that can be used to assess the fetus.

Other embodiments assign a weighted value based upon the location of the genomically unstable locus. In one embodiment, the weighted value is assigned based upon determining on which chromosome the unstable locus resides. Different chromosomes have varying functions. Instabilities in certain chromosomes lead to fetal abnormalities whereas instabilities in other chromosomes have no link to fetal abnormalities. Therefore, genomic instabilities on a certain chromosome are often indicative of a certain type of fetal abnormality, whereas genomic instabilities on other chromosomes have no link to fetal abnormalities.

An example of assigning weighted values to genomically unstable loci based upon on which chromosome the loci reside is shown here. Consider a sample in which twenty genomic instabilities are found on chromosome 14, five genomic instabilities are found on chromosome 10, and three genomic instabilities are found on each of chromosomes 4 and 9. Assume that chromosomes 14 is highly associated with fetal abnormalities; that chromosomes 9 and 10 are moderately associated with fetal abnormalities; and that chromosome 4 is not associated with fetal abnormalities. Using a scale of −10 to +10 for weighted values, genomic instabilities found on chromosome 14 are assigned a value of +10 because chromosome 14 is highly associated with fetal abnormalities and in this sample the chromosome had the highest number of genomic instabilities. Genomic instabilities found on chromosome 4 are assigned a value of −10, because instabilities found on chromosome 4 are not generally associated with fetal abnormalities. Genomic instabilities found on chromosome 9 are assigned a weighted value of 3 because chromosome 9 is associated with a fetal abnormality, however, there are only three genomic instabilities on chromosome 9 as compared to chromosome 14 that has twenty genomic instabilities. Similarly, genomic instabilities found on chromosome 10 are assigned a weighted value of +4 because chromosome 10 is associated with a fetal abnormality, however, there are only five genomic instabilities on chromosome 10 as compared to chromosome 14 that has twenty genomic instabilities. Based on different values assigned to each genomic instability, it can be determined that the fetus most likely has an abnormality associated with chromosome 14 and is potentially at risk of developing an abnormality associated with either or both of chromosomes 9 and 10.

Other embodiments assign a weighted value based upon proximity of the genomic instability to known or suspected locations of instabilities in certain fetal abnormalities. To carry out such methods, identified genomic instabilities are compared to a specific fetal abnormality reference sequences, and then weighed according to their locations in regards to the known instabilities that are associated with the specific fetal abnormalities. For example, consider that fetal abnormality X has a genomically unstable locus in the middle of chromosome A, and another genomically instable locus between chromosomes B and C. A sample has a genomically unstable locus in the middle of chromosome A, and a instability near an end of chromosome B. A high weighted value, such as a +10, will be assigned to the locus in the middle of chromosome A because such locus represents an exact match to location of an instability on chromosome X. The instability near the end of chromosome B will have a lower weighted value because it is not an exact match, however the weighted value should reflect the closeness of the genomic instability near the instability between B and C. For example, if the genomically unstable locus is 2 bases away from the fetal abnormality causing instability, its weighted value may be a +7, whereas if the genomically unstable locus is 10 bases away, the weighted value may be a +4. The weighed values may then be used to assess the sample in relation to fetal abnormality X.

Other embodiments assign a weighted value based upon proximity of the genomic instability to the telomeres. Proximity to telomeres is an important characteristic because telomeres and telomerase are linked to certain fetal abnormalities. Telomeres are responsible for regulating cell division by capping chromosomes to prevent the ends of intact chromosomes from appearing like DNA breaks to the DNA replication machinery. Telomeres functioning properly prevent chromosomal degradation, fusion, and rearrangements during DNA replication. With normal cell replication, the telomeres begin to shorten until the telomere is gone and the cell dies. However, in a fetus having a fetal abnormality, genomic instabilities may prevent the telomeres from getting shorter by initiating an enzyme called telomerase. Telomerase is found in many fetal abnormalities and allows mutated fetal cells to replicate indefinitely. The following provide more detailed description of telomeres, telomerase, and genomic instabilities De Lange, T. “Telomere-related Genome Instability in Cancer.” Cold Spring Harb. Symp. Quant. Bio. 70 (2005): 197-204, and Greider, Carol, et al. “Telomeres, Telomerase and Cancer.” Scientific American (2009). Genomic instabilities on or near telomeres may further cause various different fusions, additions, deletions, translocations all of which may contribute to fetal abnormalities. Therefore, location of instabilities near or on telomeres may provide invaluable insight towards assessing a fetus.

In determining how to weigh the genomically unstable loci near telomeres, many factors may affect the weighted value such as whether the proximity of the genomic instability to the telomere has been linked to fetal abnormalities in fetal abnormality reference sequences, the potential of the locus in impacting the telomere's function, type of instability and its proximity to the telomere, the amount of genetic material affected by the instability in regards to its proximity to the telomere, and the exact location in regards to the telomere, i.e. on the telomere, a base away from the telomere, or a few bases away from the telomere. For example, fetal abnormality X has a genomically unstable locus residing on a telomere. A sample has a genomically unstable locus two bases away from the telomere. Here, the weighted value may be a +8 because two bases is very close to the telomere and such close proximity may have the potential to impact the telomere's function. In another example, consider that genomically unstable loci located on a first telomere of chromosome A are known to be causally linked to fetal abnormality Y and that genomically unstable loci located on the second telomere of chromosome A have not yet been causally associated with fetal abnormality Y. A sample reference has a genomically unstable locus on the first telomere and a genomically unstable locus on the second telomere. The genomically unstable locus on the first telomere will have a +10 because it represents an exact match to fetal abnormality Y. The genomically unstable locus on the second telomere may have a +7, because its telomere is not yet associated with fetal abnormality Y, but telomeres perform similar functions and its location on the same chromosome may result in the instability having the same fetal abnormality causing significance.

In certain embodiments, a weighted value is assigned to a genomic instability based upon the amount of genomic material perturbed by the instability, i.e., the number of nucleotides affected by the instability. A weighted value may be assigned based upon the amount of genetic material affected in the aggregate. In this embodiment, weighted values may be assigned to proportionally reflect the amount of material affected in comparison with other locus. For example, an addition affects 4 bases whereas a translocation affects 10 bases. The weighted value for the addition will be +2 whereas the weighted value for the translocation will be +5. The weighted value of +2 for the addition and the weighted value of +5 for the translocation proportionally and comparatively represent the amount of material affected in each locus.

In another embodiment, the amount of genetic material perturbed by a locus or loci may further be characterized by subdividing the amount of genetic material affected into regions. A single genomic instability may be subdivided into regions, or all of the genomic material affected by all of the genomically unstable locus may be placed into regional categories. The regional divisions may include coding v. non-coding and introns v. exons. A weighted value may be assigned to reflect the amount of genetic material affected in each region. In an example, fetal abnormality X has a known genomically unstable locus affecting 10 nucleotides. A nucleic acid from a sample also has a genomic instability at the same genomically unstable locus that is known to be associated with fetal abnormality X, however, the genomic instability from the sample affects only 3 nucleotides. In this case, the sample genomically unstable locus is assigned a value of +3 to reflect the amount of genetic material affected in comparison to the genomically unstable locus associated with fetal abnormality X. In another example, a genomically unstable locus affects 50 bases in a non-coding region and another genomically unstable locus affects 10 bases in a coding region of chromosome Y. The non-coding region may have a value of +2 because non-coding region mutations do not affect protein function. The coding region in the same sample may have a weighted value of +6, even though less bases were affected, because its function in coding protein carries with it a higher fetal abnormality causing potential.

In certain embodiments, more than one characteristic of the genomic instability is assessed to determine the value assigned to that instability. For example, an instability can be assigned a value not only based on its type (e.g., addition, deletion, translocation), but also its proximity to a telomere and its proximity to a known fetal abnormality causing genomic instability. In one example, the weighted value for a genomically unstable locus represents the severity of the locus factoring in that the locus is an addition (type) and the addition affects multiple nucleotides (amount of genetic material affected). In such an example, the value reflects two characteristics of the locus. In another example, a weighted value represents that the locus is a gene amplification (type) affecting only a small amount of genetic material (amount of genetic material affected) on a certain chromosome (location). Such example factored in all three characteristics in determining the weighted value.

Another aspect of the invention assesses assessing a fetus by analyzing a nucleic acid from a sample, identifying one or more genomically unstable loci in the nucleic acid, categorizing the genomically unstable loci, assigning a weighted value to each category, and assessing a fetal abnormality based on the weighted values. The categories include but are not limited to the type of genomic instability, the location of the genomic instability, and the amount of genetic material affected. Applying a weighted value to a category reflects the overall influence of the category containing certain genomic characteristics within the sample.

A method of calculating a weighted sum from the weighted values of the categories is provided here. The weighted sum reflects the overall influence of all of the genomically unstable loci within the sample. A weighted sum may be devised by adding each category's weighted value times the corresponding amount of genomically unstable loci in each category or amount of genetic material affected in each category. For example, category 1 has a weighted value of 10 and contains 2 genomically unstable locus and category 2 has a weighted value of 4 and 1 genomically unstable locus. The corresponding weighted sum equals 24, the result of (10×2)+(4×1). The invention further provides for calculating a weighted average where the weighted sum is divided by the amount of genomically unstable locus in the sample. The weighted average may allow for a more manageable value in the case where weighted sums are extremely large. The weighted average using the above weighted sum equals 8 (the weighted value 24/(2 genomically unstable locus+1 genomically unstable locus).

For example, if the genomically unstable categories are based on type, one sample may include a category of deletion, a category of additions, and a category of gene amplifications. A weighted value for each category may be assigned based on the amount of genomically unstable loci in each category. For example, the weighted value is proportional to the amount of loci in the categories. Take a sample that when categorized by type results in two categories, a deletion category with 7 deletion-type genomically unstable loci and an addition category with 3 addition-type genomically unstable loci. Assigning values to the category's proportionally based on amount results in the deletion category having a weighted value of 7 and the addition category of 3. In another embodiment, a weighted value for a category may be the average of the weighted values for each individual genomically unstable locus. The weighted value for each individual genomically unstable locus is assigned based on the above embodiments of the invention. For example, after categorizing, a sample has a deletion category composed of 2 deletions, deletion A was assigned 8 and deletion B was assigned a 4. The resulting weighted value of the deletion category is 6, calculated by adding the weighted values (8+4=12) divided by the number of weighted values (2).

### Providing and Recording Targeted Therapeutic Treatment Based on Quantitative and Qualitative Assessment

Methods of this invention are useful because certain therapies may be able to eliminate or reduce the severity of the fetal abnormality. Therefore, by assessing the presence of specific genomically unstable loci in a sample and/or the quantity of genomically unstable loci, therapeutic treatment can be provided to the fetus based on the genomically unstable loci and the presence of a particular type of fetal abnormality.

An embodiment of the invention includes a reference log based upon the methods of the invention described above and includes the targeted therapeutic treatments provided to fetuses based upon the number and weighted values of genomically unstable loci in a sample and the efficacy of the therapeutic treatments for the fetus. The reference log contains a total assessment of the genomically unstable loci as compared to a reference sequence and sequences of certain fetal abnormalities. In certain embodiments, the reference sequence is a normal sequence and the fetal abnormality sequence is from a patient having the abnormality.

After the genomically unstable loci in a first sample are identified, quantified and assigned weighted values and sums based on selected characteristics and categories, the quantity of genomically unstable loci and calculated weighted values and sums are recorded in a reference log for the fetus. A second sample from the same patient is taken after a lapse in time, during a course of treatment, or after a course of treatment. Methods of the invention are performed on the second sample to identify, quantify, and assign weighted values and sums to the genomically unstable loci using the same scaling methods and the same characteristics and categories used for sample 1.

The second sample's quantity of genomically unstable loci, weighted values and sums are likewise recorded in the fetus' reference log. Variances in the quantity and corresponding weighted values and sums between the two samples represents changes in the fetal abnormality. If the second sample is taken after a course of treatment, the variances among the quantity, weighted values, and sums are indicative of whether the course of treatment is effective. Because the weighted values represent each genomically unstable locus, either individually or categorically, the course of treatment can be specifically tailored to treat genomically unstable loci that are not responding to the treatment.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of assessing a fetal abnormality, the method comprising:
analyzing a nucleic acid from a sample comprising fetal nucleic acid;
identifying one or more genomically unstable loci in the nucleic acid;
assigning a weighted value to each genomically unstable locus; and
assessing a fetal abnormality based on the weighted values.

2. The method according to claim 1, wherein the weighed value is based on severity of the genomically unstable locus.

3. The method according to claim 1, wherein the weighted value is based on a type of genomic instability.

4. The method according to claim 3, wherein the type of genomic instability is selected from the group consisting of additions, deletions, substitutions, translocations, alterations, amplifications, and single nucleotide polymorphisms.

5. The method according to claim 1, wherein the weighted value is based on a location of the genomically unstable locus.

6. The method according to claim 5, wherein the location is selected from the group consisting of: location on a chromosome, proximity to telomeres, and proximity to known or suspected locations of genomic instabilities found in certain fetal abnormalities.

7. The method according to claim 1, wherein the weighted value is based on a number of nucleotides within each genomically unstable locus.

8. The method according to claim 1, wherein analyzing comprises sequencing the nucleic acid.

9. The method according to claim 8, wherein sequencing is sequencing-by-synthesis.

10. The method according to claim 8, wherein identifying comprises comparing the sequenced nucleic acid to a reference nucleic acid to thereby identify the genomically unstable loci.

11. The method according to claim 1, wherein prior to the assigning step, the method further comprises categorizing the genomically unstable loci.

12. The method according to claim 11, further comprising deriving a weighted sum for each category.

13. The method according to claim 11, wherein categorizing is based on a type of genomic instability.

14. The method according to claim 11, wherein categorizing is based on a location of the genomically unstable locus.

15. The method according to claim 1, wherein the sample is a maternal sample.

16. The method according to claim 15, wherein the maternal sample is blood or amniotic fluid.

17. The method according to claim 1, wherein the sample is a fetal sample.
